SEQUENCE LISTING (14 sequences)

SEQ ID NO: 1 - DNA, 21 bp, Homo sapiens
aagctggactcctcctacaca

SEQ ID NO: 2 - DNA, 21 bp, Homo sapiens
aaaggaatgacttccagctga

SEQ ID NO: 3 - DNA, 21 bp, Artificial Sequence
  Synthetic oligonucleotide
acacauccuccucaggucgtt

SEQ ID NO: 4 - DNA, 21 bp, Artificial Sequence
  Synthetic oligonucleotide
cgaccugaggaggaugugutt

SEQ ID NO: 5 - DNA, 21 bp, Homo sapiens
gcuggacuccuccuacacatt

SEQ ID NO: 6 - DNA, 21 bp, Artificial Sequence
  Synthetic oligonucleotide
uguguaggaggaguccagctt

SEQ ID NO: 7 - DNA, 1309 bp, Homo sapiens
  CDS (122)..(586)
ggcacgagggtagaggcggcggcggcggcggcagcgggctcggaggcagcggttgggctcgcggcgagcggacggggtcgagtcagtgcgttcgcgcgagttggaatcgaagcctcttaaaatggcagatgacttggacttcgagacaggagatgcaggggcctcagccaccttcccaatgcagtgctcagcattacgtaagaatggctttgtggtgctcaaaggccggccatgtaagatcgtcgagatgtctacttcgaagactggcaagcacggccacgccaaggtccatctggttggtattgacatctttactgggaagaaatatgaagatatctgcccgtcaactcataatatggatgtccccaacatcaaaaggaatgacttccagctgattggcatccaggatgggtacctatcactgctccaggacagcggggaggtacgagaggaccttcgtctccctgagggagaccttggcaaggagattgagcagaagtacgactgtggagaagagatcctgatcacggtgctgtctgccatgacagaggaggcagctgttgcaatcaaggccatggcaaaataactggctcccaggatggcggtggtggcagcagtgatcctctgaacctgcagaggccccctccccgagcctggcctggctctggcccggtcctaagctggactcctcctacacaatttatttgacgttttattttggttttccccaccccctcaatctgtcggggagcccctgcccttcacctagctcccttggccaggagcgagcgaagctgtggccttggtgaagctgccctcctcttctcccctcacactacagccctggtgggggagaagggggtgggtgctgcttgtggtttagtctttttttttttttttttttttttttaaattcaatctggaatcagaaagcggtggattctggcaaatggtccttgtgccctccccactcatccctggtctggtcccctgttgcccatagccctttaccctgagcaccaccccaacagactggggaccagccccctcgcctgcctgtgtctctccccaaacccctttagatggggagggaagaggaggagaggggaggggacctgccccctcctcaggcatctgggagggccctgcccccatgggctttacccttccctgcgggctctctccccgacacatttgttaaaatcaaacctgaataaaactacaagtttaatatgaaaaaaaaaaaaaaaaaaaaaaaaaaaaaaaaaaaaaaaaaaaaaaaaaaaaaaaaaaaaaaaaaaaaaaaaaa

SEQ ID NO: 8 - protein, 154 aa, Homo sapiens
MADDLDFETGDAGASATFPMQCSALRKNGFVVLKGRPCKIVEMSTSKTGKHGHAKVHLVGIDIFTGKKYEDICPSTHNMDVPNIKRNDFQLIGIQDGYLSLLQDSGEVREDLRLPEGDLGKEIEQKYDCGEEILITVLSAMTEEAAVAIKAMAK

SEQ ID NO: 9 - DNA, 447 bp, Homo sapiens
ttcgagacaggagatgcaggggcctcagccaccttcccaatgcagtgctcagcattacgtaagaatggctttgtggtgctcaaaggccggccatgtaagatcgtcgagatgtctacttcgaagactggcaagcacggccacgccaaggtccatctggttggtattgacatctttactgggaagaaatatgaagatatctgcccgtcaactcataatatggatgtccccaacatcaaaaggaatgacttccagctgattggcatccaggatgggtacctatcactgctccaggacagcggggaggtacgagaggaccttcgtctccctgagggagaccttggcaaggagattgagcagaagtacgactgtggagaagagatcctgatcacggtgctgtctgccatgacagaggaggcagctgttgcaatcaaggccatggcaaaataa

SEQ ID NO: 10 - protein, 148 aa, Homo sapiens
FETGDAGASATFPMQCSALRKNGFVVLKGRPCKIVEMSTSKTGKHGHAKVHLVGIDIFTGKKYEDICPSTHNMDVPNIKRNDFQLIGIQDGYLSLLQDSGEVREDLRLPEGDLGKEIEQKYDCGEEILITVLSAMTEEAAVAIKAMAK

SEQ ID NO: 11 - DNA, 19 bp, Homo sapiens
gctggactcctcctacaca

SEQ ID NO: 12 - RNA, 19 nt, Homo sapiens
gcuggacuccuccuacaca

SEQ ID NO: 13 - DNA, 3370 bp, Artificial Sequence
  Plasmid pExp5A
ttaattaaaattatctctaaggcatgtgaactggctgtcttggttttcatctgtacttcatctgctacctctgtgacctgaaacatatttataattccattaagctgtgcatatgatagatttatcatatgtattttccttaaaggatttttgtaagaactaattgaattgatacctgtaaagtctttatcacactacccaataaataataaatctctttgttcagctctctgtttctataaatatgtacaagttttattgtttttagtggtagtgattttattctctttctatatatatacacacacatgtgtgcattcataaatatatacaatttttatgaataaaaaattattagcaatcaatattgaaaaccactgatttttgtttatgtgagcaaacagcagattaaaaggaattctcgagtcatcgatactagtgcgaccgccaaaccttagcggcccagctgacaaaagcctgccctcccccagggtccccggagagctggtgcctcccctgggtcccaatttgcatggcaggaaggggcctggtgaggaagaggcggggaggggacaggctgcagccggtgcagttacacgttttcctccaaggagcctcggacgttgtcaagcttctgccttctccctcctgtgagtttggtaagtcactgactgtctatgcctgggaaagggtgggcaggagatggggcagtgcaggaaaagtggcactatgaaccctgcagccctaggaatgcatctagacaattgtactaaccttcttctctttcctctcctgacaggttggtgtacagtagcttccaccatggcagatgatttggacttcgagacaggagatgcaggggcctcagccaccttcccaatgcagtgctcagcattacgtaagaatggttttgtggtgctcaagggccggccatgtaagatcgtcgagatgtctacttcgaagactggcaggcatggccatgccaaggtccatctggttggtattgatatttttactgggaagaaatatgaagatatctgcccgtcgactcataacatggatgtccccaacatcaaaaggaatgatttccagctgattggcatccaggatgggtacctatccctgctccaggacagtggggaggtacgagaggaccttcgtctgcctgagggagaccttggcaaggagattgagcagaagtatgactgtggagaagagatcctgatcacagtgctgtccgccatgacagaggaggcagctgttgcaatcaaggcgatggcaaaataactggctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggcggccgcaccaccctgggccaggctgggccaagccaggcggcccctgtgttttccccagtctctgggctgctggagggaaccaggttgttttggcatcagcctctactgagccggagcccttcctttcctgctgctttgcatagtggcactaattccgtcctcctacctccaccagggacctaggcagccgggtagatggtgggaggaggcttcacttctcccccaagcagggtctccacctgcttgaggctgccctgggttgggggaggccttggctttacctaaagactttttaacacctctacgcgtaattcagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatgatctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcaagacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcttaattaaaacaggtagttgacaattaaacattggcatagtatatctgcatagtataatacaactcactataggagggccatcatggccaagttgaccagtgctgtcccagtgctcacagccagggatgtggctggagctgttgagttctggactgacaggttggggttctccagagattttgtggaggatgactttgcaggtgtggtcagagatgatgtcaccctgttcatctcagcagtccaggaccaggtggtgcctgacaacaccctggcttgggtgtgggtgagaggactggatgagctgtatgctgagtggagtgaggtggtctccaccaacttcagggatgccagtggccctgccatgacagagattggagagcagccctgggggagagagtttgccctgagagacccagcaggcaactgtgtgcactttgtggcagaggagcaggactgaggataacctaggaaaccttaaaacctttaaaagccttatatattcttttttttcttataaaacttaaaaccttagaggctatttaagttgctgatttatattaattttattgttcaaacatgagagcttagtacatgaaacatgagagcttagtacattagccatgagagcttagtacattagccatgagggtttagttcattaaacatgagagcttagtacattaaacatgagagcttagtacatactatcaacaggttgaactgctgatc

SEQ ID NO: 14 - protein, 154 aa, Artificial Sequence
  Amino Acid Sequence of eIF5A1K50R
MADDLDFETGDAGASATFPMQCSALRKNGFVVLKGRPCKIVEMSTSKTGRHGHAKVHLVGIDIFTGKKYEDICPSTHNMDVPNIKRNDFQLIGIQDGYLSLLQDSGEVREDLRLPEGDLGKEIEQKYDCGEEILITVLSAMTEEAAVAIKAMAK

(19) United States
(12) Patent Application Publication
(10) Pub. No.: US 2014/0314704 A1
(43) Pub. Date: Oct. 23, 2014

(54) Treatment of B Cell Lymphomas

(71) Applicant: Senesco Technologies, Inc., Bridgewater, NJ (US)

(72) Inventor: John E. Thompson, Waterloo (CA)

(21) Appl. No.: 14/362,005

(22) PCT Filed: Nov. 30, 2012
(86) PCT No.: PCT/US2012/067330

§ 371 Date: May 30, 2014

Related U.S. Application Data
(60) Provisional application No. 61/565,418, filed on Nov. 30, 2011.

(51) Int. Cl. A61K 47/48; A61K 31/69; A61K 31/454; A61K 31/713; A61K 48/00

(52) U.S. Cl. CPC A61K 47/48207; A61K 31/713; A61K 48/005; A61K 31/454; A61K 31/69
USPC 424/78.29; 514/44 A

(57) ABSTRACT

Controlled-release formulations of carboxy-terminal C5a analogs (such as sustained-release formulations of the analogs), and their use in methods for treating and preventing an infection or a disease such as cancer, for directly killing microorganisms, for vaccine preparation, for inducing an immune response and for targeting antigen-presenting cells and other cells bearing a C5a receptor, are provided.

### BACKGROUND

The present invention relates to methods of treating B cell lymphomas by manipulation of eIF-5A1 expression. The use of a composition comprising an siRNA targeted against the eIF-5A1 gene to suppress endogenous expression of this gene in a subject, and a polynucleotide encoding a mutant eIF-5A1 capable of being expressed in the subject for the treatment of multiple myeloma has been previously discussed (see U.S. 20100076062 and 20100004314).

### SUMMARY OF THE INVENTION

The invention relates to the treatment of B cell lymphomas by administration of siRNA which blocks expression of endogenous eIF-5A1 in combination with an expression plasmid which provides for the expression of eIF-5A1 which cannot be hypusinated in the subject.

The invention further provides for the treatment of multiple myeloma by co-administration of siRNA which blocks expression of endogenous eIF-5A1 with an expression plasmid which provides for the expression of eIF-5A1 which cannot be hypusinated in combination with bortezomib or lenalidomide.

### BRIEF DESCRIPTION OF DRAWINGS

FIG. 1: SNS01-T dose response curve in mice with DLBCL tumors.

FIG. 2: SNS01-T dose response curve in mice with DLBCL tumors.

FIG. 3: SNS01-T with truncated eIF5A dose response curve in mice with DLBCL tumors.

FIG. 4: Mouse body weight following administration of SNS01-T.

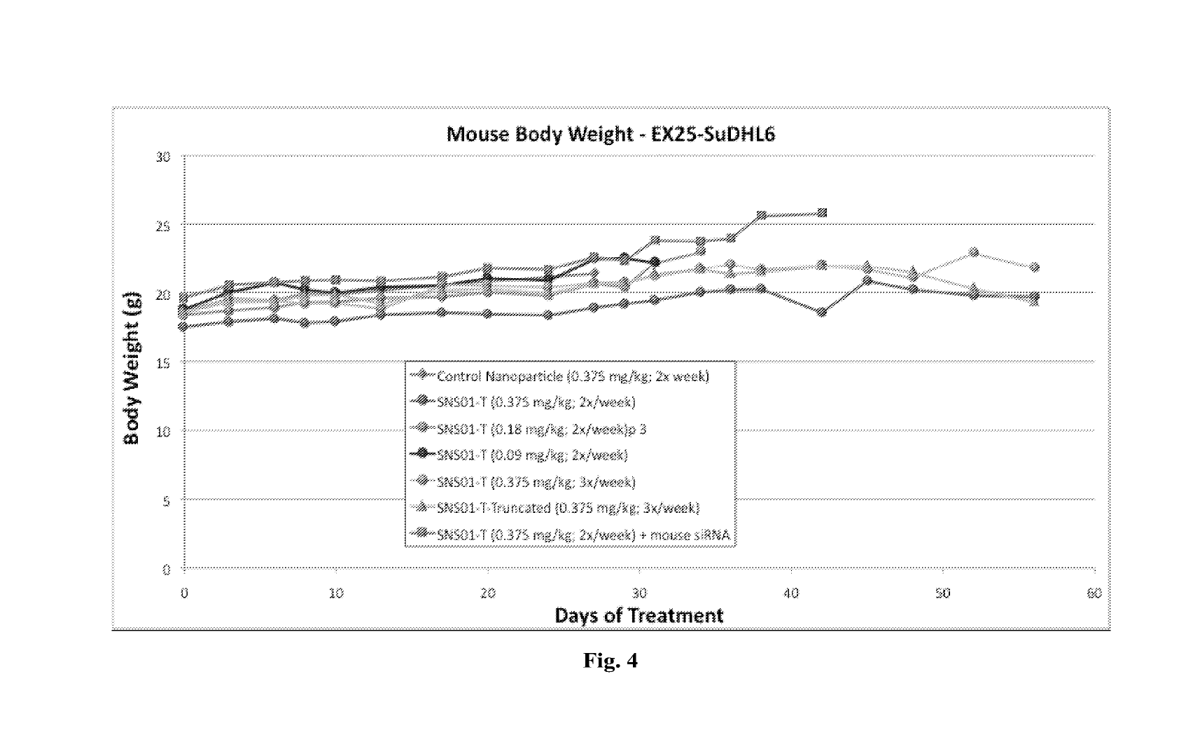

FIG. 5: SNS01-T dose response curve in mice with mantle cell lymphoma tumors.

FIG. 6: SNS01-T treatment of mantle cell lymphoma tumor in combination with Lenalidomide.

FIG. 7: Map of SNS01-T eIF-5A expression plasmid (SEQ ID NO: 13).

### DETAILED DESCRIPTION

The present invention provides methods for treating B cell lymphomas such as, for example, diffuse large B cell lymphoma (DLBCL), chronic lymphocytic leukemia (CLL), mantle cell lymphoma and follicular lymphoma. In some embodiments, the invention encompasses selecting a patient suffering from a B cell lymphoma for the treatment methods described herein.

The invention provides a method of treating B cell lymphomas comprising administering a composition comprising a complex of an eIF-5A1 siRNA targeted against the 3′ end of eIF-5A1, an expression vector comprising a polynucleotide encoding a mutant eIF5A1 wherein the mutant eIF5A1 is unable to be hypusinated, wherein the siRNA and the expression vector are complexed to polyethylenimine to form a complex.

As the invention provides use of a composition comprising an siRNA targeted against a target gene to suppress endogenous expression of the target gene is a subject, and a polynucleotide encoding a target protein capable of being expressed in the subject for the treatment of B cell lymphomas, in certain embodiments the polynucleotide is in RNAi resistant plasmid (will not be suppressed by the siRNA).

In certain embodiments the siRNA targets the eIF-5A1 sequence shown in SEQ ID NO: 1 and the polynucleotide encoding the mutant eIF-5A1 is eIF-5A1_{K50R}. The expression vector comprises a polynucleotide encoding a mutant eIF5-A1 and a promoter operably linked to provide expression of the polynucleotide in a subject. The promoter preferably is either tissue specific or ubiquitous. For example, if the composition is used to treat B cell lymphoma, the promoter is tissue specific for the tissue in which the cancer resides. The promoter can therefore be specific for tissues where B cells are normally found, such as bone marrow and lymphoid tissue (i.e. lymph nodes and spleen), or can be specific for tissues where B cells do not normally reside but where a B cell tumor has formed, such as the lung or other tissue sites located in the diaphragm. For example, for treating B cell lymphoma, it is preferable to use a B cell specific promoter, such as B29. In certain embodiments, the expression vector comprises a pCpG plasmid.

The present invention also provides for methods of treating B cell lymphomas using an isolated polynucleotide encoding a truncated form of eIF-5A1 as well as a truncated eIF-5A1 polypeptide. The truncated eIF-5A1 polynucleotide is useful in inducing apoptosis and killing cancer cells. The truncated polynucleotide may be used within an expression vector which is then administered to a mammal. The truncated eIF-5A form is expressed within the mammal and kills cancer cells. The truncated eIF-5A1 protein is about 16 kDA as opposed to the full length elf-5A1 protein, which is about 17 kDa.

In certain embodiments the truncated eIF-5A1 polynucleotide comprises or consists of the sequence set forth in SEQ ID NO: 9 and the amino acid sequence comprises or consists of SEQ ID NO: 10. In certain embodiments the truncated eIF-5A1 polynucleotide is comprised within a plasmid or expression vector. Plasmids and expression vectors are described herein below in more detail. In certain embodiments the expression vector is an adenovirus expression vector or is pHM6. In certain embodiments the expression vector comprises a tissue specific promoter, such as a B cell specific promoter (i.e. B29) when the composition or medicament is used to treat multiple myeloma. The expression vector may comprise a pCpG plasmid. As discussed in more detail hereinbelow, the expression vector may be complexed to polyethylenimine.

In certain embodiments, the eIF-5A1 siRNA and the expression vector comprising the mutant eIF-5A1 polynucleotide are independently complexed to polyethylenimine, such as in vivo JET-PEI. In other embodiments, the eIF-5A1 siRNA and the expression vector comprising the mutant eIF5-A1 polynucleotide are complexed together to polyethylenimine.

The present invention further provides a method for treating B cell lymphoma in a subject in need thereof comprising administering a composition comprising an eIF-5A1 siRNA targeted against the 3′ end of eIF-5A1 gene and an expression vector comprising a polynucleotide encoding a mutant eIF-5A1, wherein the mutant eIF5A1 is unable to be hypusinated. The B cell lymphoma can be any of diffuse large B cell lymphoma (DLBCL), chronic lymphocytic leukemia (CLL), mantle cell lymphoma and follicular lymphoma.

The compositions containing the siRNA and plasmids described herein may be administered via any suitable means, including, for example, parenterally, transdermally, intranasally and orally. Suitable formulations for delivery include, but are not limited to, intravenous or intramuscular administration. In some embodiments the formulation contains a lipsome while in others it contains a nanoparticle. In some embodiments, the nanoparticle is a polyethylenimine (PEI) nanoparticle.

The polynucleotide encoding a mutated eIF-5A1 is preferably mutated so that it cannot be hypusinated and thus will not be available to drive the cell into survival mode. For example, in one embodiment, the polynucleotide encoding eIF-5A is mutated to so that the lysine (K) at position 50, which is normally hypusinated by DHS, is changed to an alanine (A) or arginine (which cannot be hypusinated). This mutant is denoted as K50A or K50R, respectively.

In another embodiment, the lysine at position 67 in eIF-5A1 is changed to an arginine (R). This mutant is denoted as (K67R). In another embodiment the lysine (K) at position 67 is changed to an alanine (A) and is denoted as (K67A). In another embodiment, a mutant where the lysine (K) at position 47 is changed to an arginine (K47R) is contemplated.

In other embodiments, a double mutant of eIF-5A1 is used. One double mutant is where the lysine (K) at position 50 is changed to an arginine (R) and the lysine (K) at position 67 is changed to a arginine (R). This double mutant is referred to as K50R/K67R. This double mutant is similarly unable to be hypusinated but the changes in the amino acids do not alter the three dimensional structure of eIF-5A1 as much as the single mutation (K50A). The double mutation thus provides a protein that is very similar in 3-D shape and folding as the wild type and thus is more stable than the single mutant. Being more stable, it exists longer in the body to provide longer therapeutic benefit. Thus, the body will have the eIF-5A it needs for normal cell function but it will not be able to hypusinated so the cells do not get locked into the cell survival mode and escape apoptosis.

As the body needs eIF-5Aa for normal cell survival and healthy cell proliferation, it is preferable not to shut off expression completely in the subject with the siRNA, such as when the siRNA is delivered systemically. Control of eIF-5A expression can be achieved by either using an siRNA that does not completely eliminate expression (i.e. reduces expression but does not completely shut off expression) or alternatively, utilizing a dosing and/or treatment regimen to balance expression levels to allow normal growth and functioning of healthy cells but also to force cancerous cells to apoptosis.

Alternatively, one may utilize local delivery of siRNA. If the siRNA is delivered locally to the cancer cell or tumor, then the expression is preferably knocked out. By knocking out expression, there is no eIF-5A1 around that can be hypusinated and thus there is no hypusinated eIF-5A1 to lock the cells into survival mode. Since the siRNA is delivered locally to the cancer or tumor, there is no need to have eIF-5A available for regular cell growth.

In certain embodiments, the siRNA consists essentially of the siRNA construct shown in SEQ ID NO: 5 and 6. For example, the siRNA contains nucleic acids targeted against the eIF-5A1 but also contains overhangs such as U or T nucleic acids or also contains tags, such as a his tag (often referred to as HA tag, which is often used in in vitro studies). Molecules or additional nucleic acids attached at either the 5′ or 3′ end (or even within the consecutive string of nucleic acids) may be included and fall within the scope of the invention as long as the siRNA construct is able to reduce expression of the target gene. Preferably the siRNA targets regions of the eIF-5A1 gene so as to not effect expression of the exogenous polynucleotide. For example the eIF-5A1 siRNA targets the 3′ UTR or the 3′ end. The siRNA shown in SEQ ID NO: 5 and 6 is an exemplary eIF-5A1 siRNA.

In some embodiments, the polynucleotide encoding eIF-5A1 is mutated to encode an eIF5A1 variant. The mutated eIF-5A1 is designed so that the variant eIF-5A1 cannot be post translationally modified (i.e. cannot be hypusinated). Exemplary mutants are discussed herein above.

The methods of the present invention also encompass the administration of a polynucleotide encoding the eIF-5A2 isoform (GenBank Accession number NM 020390). eIF-5A2 isoform induces apoptosis in cancer cells when expressed (see U.S. 20070154457). The present invention thus provides methods for treating B cell lymphomas such as, for example, diffuse large B cell lymphoma (DLBCL), chronic lymphocytic leukemia (CLL), mantle cell lymphoma and follicular lymphoma by administering a polynucleotide which results in expression of the eIF-5A2 isoform in the cancer cell, thereby inducing apoptosis. The eIF-5A2 polynucleotide may be delivered in a plasmid, vector, such as an adenovirus vector, or any suitable expression vector.

In another aspect, the present invention provides a method of treating B cell lymphomas or multiple myeloma in a subject (e.g. human) by administering a therapeutically effective amount of an siRNA targeted against an endogenous eIF-5A1 gene to knock out or knock down expression of the endogenous eIF-5A1 gene in a subject and delivery of a polynucleotide encoding the eIF-5A1 protein or portion thereof as described herein in combination with bortezomib (VELCADE) to a subject diagnosed with multiple myeloma. In certain embodiments, the therapeutically effective amount of bortezomib ranges from 0.5 mg/m² to 3 mg/m². In certain embodiments, therapeutically effective amount of bortezomib is approximately 1.3 mg/m². I n certain embodiments, the therapeutically effective amount of amount of an siRNA targeted against an endogenous eIF-5A1 gene to knock out or knock down expression of the endogenous eIF-5A1 gene in a subject and delivery of a polynucleotide encoding the eIF-5A1 protein or portion thereof is as described herein. In certain embodiments, the amount of an siRNA targeted against an endogenous eIF-5A1 gene to knock out or knock down expression of the endogenous eIF-5A1 gene in a subject and delivery of a polynucleotide encoding the eIF-5A1 protein or portion thereof is administered twice or thrice weekly, and the bortezomib is administered twice a week. In some embodiments, the invention encompasses selecting a human subject suffering from multiple myeloma and already receiving treatment with bortezomib for the treatment methods described herein.

In another aspect, the present invention provides a method of treating multiple myeloma in a subject (e.g. human) by administering a therapeutically effective amount of an siRNA targeted against an endogenous eIF-5A1 gene to knock out or knock down expression of the endogenous eIF-5A1 gene in a subject and delivery of a polynucleotide encoding the eIF-5A1 protein as described herein in combination with lenalidomide (REVLIMID) to a subject diagnosed with multiple myeloma. In certain embodiments, the therapeutically effective amount of lenalidomide ranges from 5 mg to 30 mg daily. In certain embodiments, therapeutically effective amount of lenalidomide is approximately 25 mg daily. In certain embodiments, the therapeutically effective amount of amount of an siRNA targeted against an endogenous eIF-5A1 gene to knock out or knock down expression of the endogenous eIF-5A1 gene in a subject and delivery of a polynucleotide encoding the eIF-5A1 protein is 0.1 mg/kg to 0.5 mg/kg. In certain embodiments, the amount of an siRNA targeted against an endogenous eIF-5A1 gene to knock out or knock down expression of the endogenous eIF-5A1 gene in a subject and delivery of a polynucleotide encoding the eIF-5A1 protein or portion thereof is administered twice or thrice weekly, and the lenalidomide is administered up to five times a week. In some embodiments, the invention encompasses selecting a human subject suffering from multiple myeloma and already receiving treatment with lenalidomide for the treatment methods described herein.

### EXAMPLES

### Example 1

### SNS01-T Composition

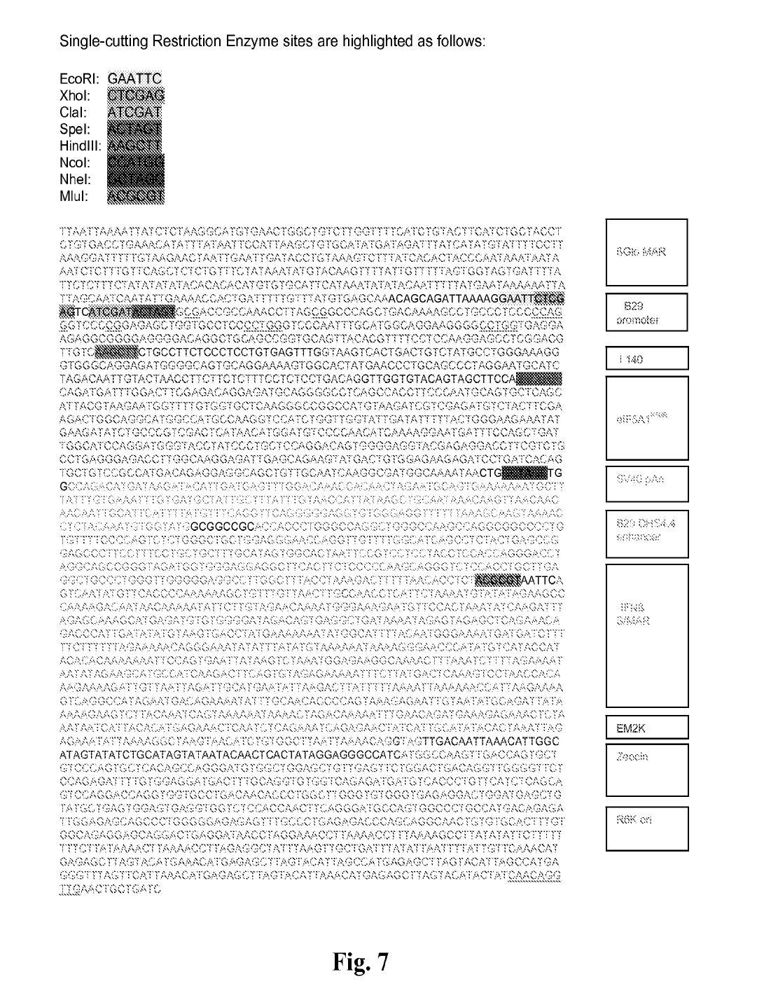

The plasmid for expression of mutant eIF-5A1_{K50R} (pExp5A) is as set forth in FIG. 7 (SEQ ID NO: 13). An expression plasmid with reduced CpG dinucleotides designed to drive expression of human eIF5A1^{K50R} (SEQ ID NO: 14) predominantly in cells of B cell lineage. The vector is derived from pCpG-LacZ, a plasmid completely devoid of CpG dinucleotides. All the elements required for replication and selection in E. coli are free of CpG dinucleotides. The original CMV enhancer/promoter and LacZ gene from the CpG-LacZ vector have been replaced with a human minimal B cell specific promoter (B29/CD79b, Invivogen) and human eIF5A1K50R, respectively, in order to drive B-cell specific expression of eIF5A1K50R. The B29 DHS4.4 3′ enhancer has been introduced into the plasmid downstream of the eIF5A1 expression cassette in order to enhance activity of the B29 promoter and reduce expression in non-B cells (Malone (2006) J. Mol. Biol. 362: 173-183). Incorporation of the B29 minimal promoter, eIF5A1K50R, and the B29 DHS4.4 3′ enhancer has introduced 32 CpG dinucleotides into the vector.

siRNA targeting human eIF-5A1: eIF5A1 siRNA target #1 (the siRNA targets this region of human eIF5A1: 5′-AAGCTGGACTCCTCCTACACA-3′ (SEQ ID NO: 1). The siRNA sequence is often referred to herein as h5A1 and is shown in SEQ ID NO: 5 and 6. eIF5A1 siRNA target #2 eIF5A1 (this siRNA targets this region of human eIF5A1: 5′-AAAGGAATGACTTCCAGCTGA-3′ (SEQ ID NO: 2). (The siRNA sequence is often referred to herein ash5A1-ALT). Control siRNA are shown in SEQ ID NO: 3 and 4.

For preparation 1 mL SNS01-T in microfuge tube, add 21.8 μL of 2.3 mg/mL pExp5A to a sterile eppendorf tube. Add 25 μL of siRNA to the eppendorf tube containing the pDNA. Use the pipette tip to gently mix by pipetting up and down slowly 5 times. Add 203 μL of 11.3 mM Tris-HCl pH 7.4 to the DNA/siRNA mixture. Use the pipette tip to gently mix by pipetting up and down slowly 5 times. Add 250 μL of 10% glucose. Use the pipette tip to gently mix by pipetting up and down slowly 10-12 times. Set tube aside and proceed to the next step. Add 9 μL of invivo-jetPEI to separate sterile eppendorf tube. Add 241 μL of 11.3 mM Tris-HCl pH 7.4 to the eppendorf tube containing the invivo-jetPEI. Use the pipette tip to gently mix by pipetting up and down slowly 5 times. Add 250 μL of 10% glucose to the invivo-jetPEI/tris mixture. Use the pipette tip to gently mix by pipetting up and down slowly 10-12 times. Using a P1000 set to 10000 μL volume, transfer the entire volume from the tube containing the DNA/siRNA/tris/glucose mixture from Step A to the tube containing the PEI/tris/glucose mixture from Step B. Use the pipette tip to gently mix by pipetting up and down slowly 10-12 times. Set tube aside at room temperature and allow a minimum of 30 minutes for nanoparticle formation prior to use.

### Example 2

### DLBCL Study Design

Female CB17-SCID mice of 3-4 weeks of age were inoculated subcutaneously with 1.2×10⁷ diffuse large cell B cell lymphoma SU-DHL6 cells. Treatment protocol as set forth below was not initiated until tumors had reached 50 mm³ while treatments were administered 2 or 3 times a week up to 6 weeks.

| | | | | # Injections | |
|---|---|---|---|---|---|
| Group # | Treatment Article | Dose | Dosing Volume | per week | n |
| 1 | Control nanoparticle | 0.375 mg/kg | 5 mL/kg (i.v.) | 2 | 5 |
| 2 | SNS01-T | 0.375 mg/kg | 5 mL/kg (i.v.) | 2 | 5 |
| 3 | SNS01-T | 0.1875 mg/kg | 2.5 mL/kg (i.v.) | 2 | 5 |
| 4 | SNS01-T | 0.093 mg/kg | 1.25 mL/kg (i.v.) | 2 | 5 |
| 5 | SNS01-T | 0.375 mg/kg | 5 mL/kg (i.v.) | 3 | 5 |
| 6 | SNS01-T-truncated | 0.375 mg/kg | 5 mL/kg (i.v.) | 3 | 5 |
| 7 | SNS01-T/mouse | 0.375 mg/kg | 5 mL/kg (i.v.) | 2 | 5 |
| | siRNA | (SNS01-T) | | | |
| | | 1 mg/kg | 1 mL/kg (i.p.) | 2 | |
| | | (siRNA) | | | |

### Example 3

### SNS01-T Dose Response in DLBCL

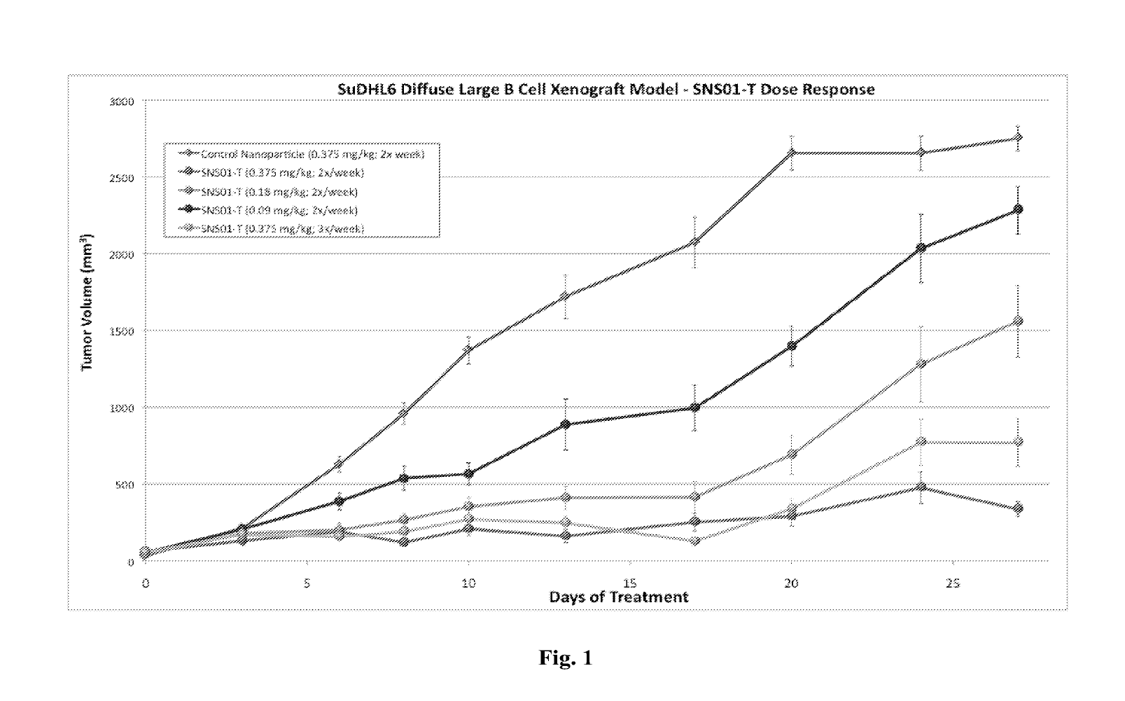

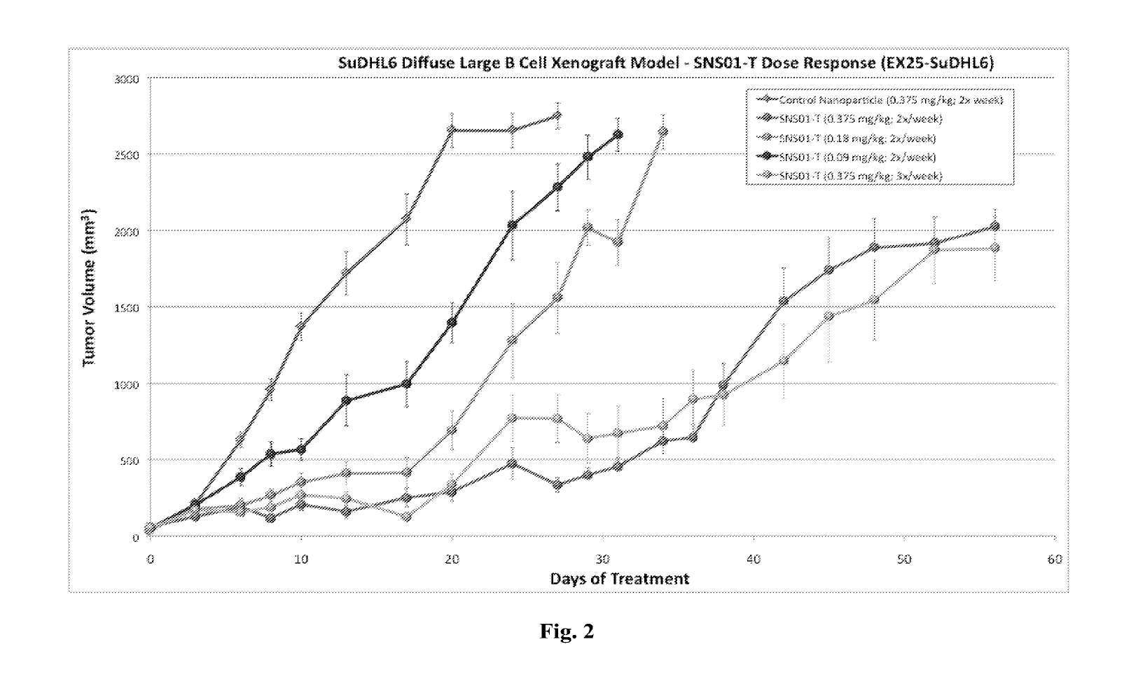

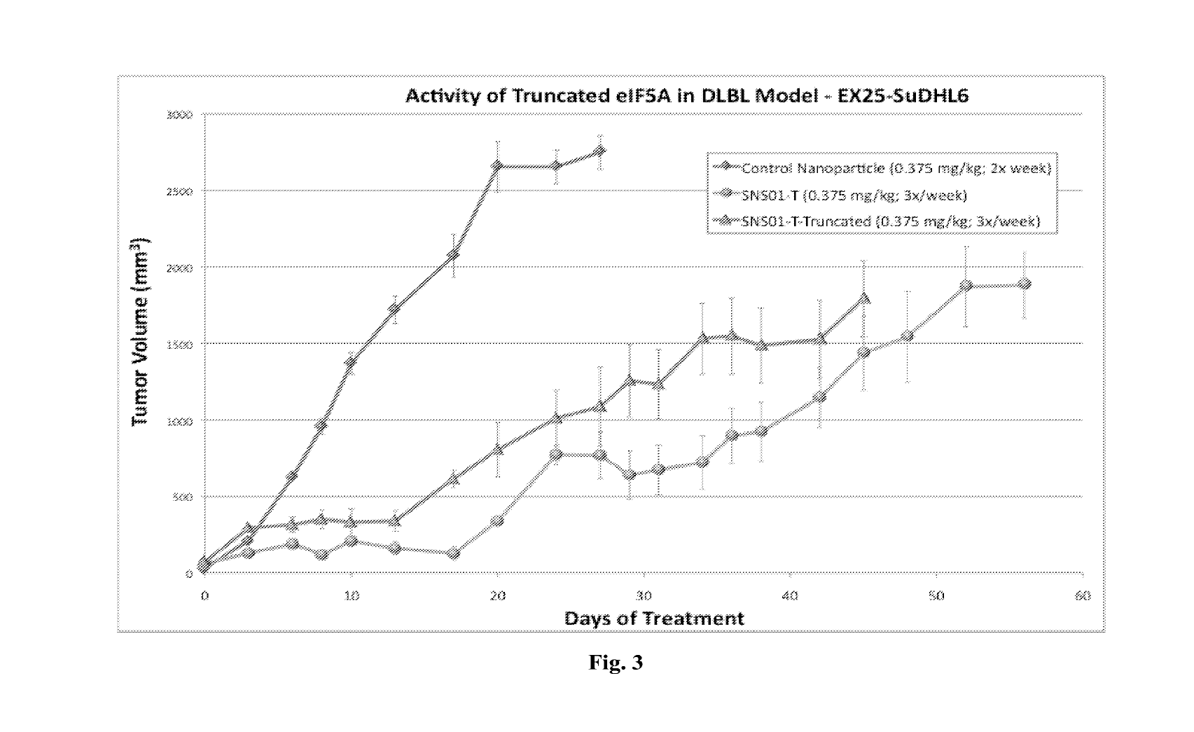

SNS01-T dose response observed no difference between twice weekly versus thrice weekly dosing (see FIG. 1-2). Tumor growth increased after end of treatment (day 38). Substituting truncated eIF5A plasmid (see FIG. 3) for pExp5A did not improve response but was still active against the DLBCL tumors (note: one mouse from this group was cured). No effect on mouse body weight was observed. Median Survival Rates in mice with DLBCL tumors treated with SNS01-T are set forth below.

Median Survival - EX25-SuDHL6

| | | | | Median | Percent |
|---|---|---|---|---|---|
| | | Dose | Total # | Survival | Increase in |
| Group | Test Article | (mg/kg) | of mice | (Days) | Survival |
| 1 | Control | 0.375 | 5 | 20.0 | 0 |
| | Nanoparticle | | | | |
| | (2x/week) | | | | |
| 2 | SNS01-T | 0.375 | 5 | 45.0 | 125% |
| | (2x/week) | | | | |
| 3 | SNS01-T | 0.188 | 5 | 29.0 | 45% |
| | (2x/week) | | | | |
| 4 | SNS01-T | 0.093 | 5 | 24.0 | 20% |
| | (2x/week) | | | | |
| 5 | SNS01-T | 0.375 | 5 | 45.0 | 125% |
| | (3x/week) | | | | |
| 6 | SNS01-T- | 0.375 | 5 | 34.0 | 70% |
| | Truncated | | | | |
| | (3x/week) | | | | |
| 7 | SNS01-T | 0.375/1 | 5 | 36.0 | 80% |
| | mouse siRNA | | | | |
| | (2x/week) | | | | |

### Example 4

### Mantle Cell Lymphoma Study Design

Female CB17-SCID mice of 3-4 weeks of age were inoculated subcutaneously with 2.5×10⁶ mantle cell lymphoma MCL-JMV-2 cells. Treatment protocol as set forth below was not initiated until tumors had reached 50 mm³ while treatments were administered 2 or 3 times a week (5 times for Lenalidomide) up to 3 weeks.

| | | | | | | # Injections | |
|---|---|---|---|---|---|---|---|
| Group # | Treatment | Dose | | Dosing Volume | | per week | n |
| 1 | Control | 0.375 | mg/kg | 5 | mL/kg/i.v. | 2 | 5 |
| 2 | SNS01-T | 0.75 | mg/kg | 10 | mL/kg/i.v. | 2 | 5 |
| 3 | SNS01-T | 0.375 | mg/kg | 5 | mL/kg/i.v. | 2 | 5 |
| 4 | SNS01-T | 0.1875 | mg/kg | 2.5 | mL/kg | 2 | 5 |
| 5 | SNS01-T | 0.093 | mg/kg | 1.25 | mL/kg | 2 | 5 |
| 6 | Lenalidomide | 15 | mg/kg | 5 | mL/kg/i.p. | 5 | 5 |
| 7 | SNS01-T & | 0.375 | mg/kg | 5 | mL/kg/i.v. | 2 | 5 |
| | Lenalidomide | 15 | mg/kg | 5 | mL/kg/i.p. | 5 | |
| 8 | SNS01-TFF | 0.75 | mg/kg | 10 | mL/kg/i.v. | 2 | 5 |

### Example 5

### SNS01-T in Mantle Cell Lymphoma

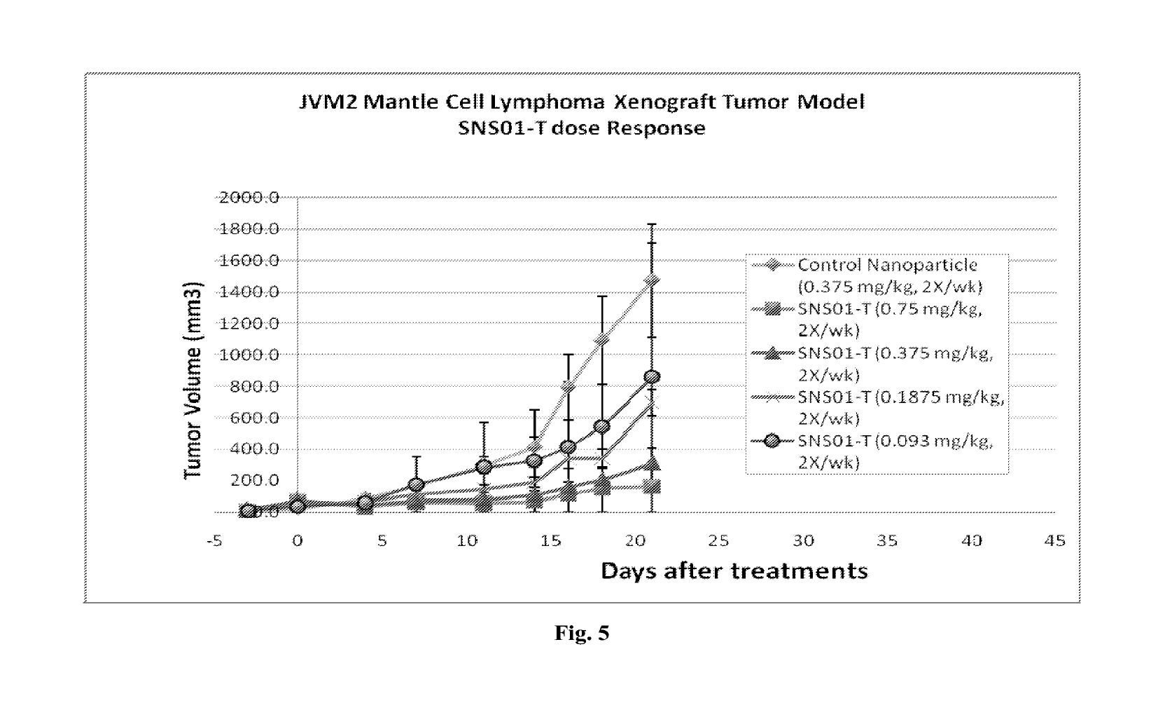

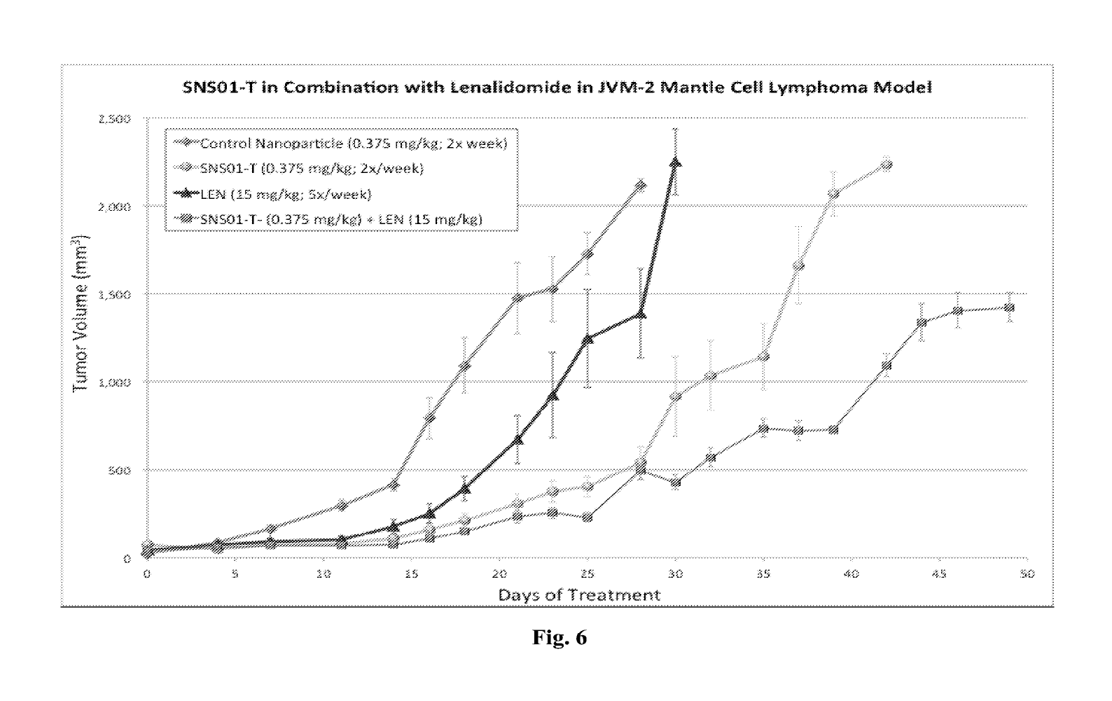

SNS01-T dose response demonstrated dependent reduction in tumor size (see FIG. 5). SNS01-T and lenalidomide combination drug therapy is more effective than monotherapy in controlling growth of mantle cell lymphoma xenograft tumors (FIG. 6). SCID mice were implanted with 0.25×10⁶ JVM-2 MCL cells s into the right flank. Treatment was initiated when the tumors reached an average size of 50 mm³ Mice were treated twice weekly (3-4 days between injections) with either control nanoparticles or SNS01-T at 0.375 mg/kg. Mice receiving lenalidomide (LEN) treatment received intra-peritoneal injections of 15 mg/kg 5 times per week. Tumor dimensions were measured 2-3 times weekly. Treatment continued for 51 days. Data shown is mean tumor volume±standard error (* p<0.05, ** p<0.01, *** p<0.001 compared to control group).

### Example 6

### Treatment of Multiple Myeloma with SNS01-T with Lenalidomide or Bortezomib

There is additional benefit to combining SNS01-T with other approved multiple myeloma drugs such as lenalidomide or bortezomid. We have shown that these drugs work up to approximately 40 times more effectively when used in combination with SNS01-T.

| | IC₅₀ | IC₅₀ with IC₂₀ SNS01-T |
|---|---|---|
| Cell lines | Bortezomib, nM | Bortezomib, nM |
| KAS-6/1 | 7.31 | 2.64 (~2.8× less) |
| U266 | 2.86 | 0.83 (~3.4× less) |
| RPMI-8226 | 2.12 | 1.51 (~1.4× less) |
| | Lenalidomide, μM | Lenalidomide, μM |
| KAS-6/1 | 69.78 | 2.37 (~29× less) |
| U266 | 62.05 | 1.42 (~43.7× less) |
| RPMI-8226 | 84.94 | 3.28 (25.9× less) |

1. A method of treating a B cell lymphoma in a human subject in need thereof comprising administering:
(a) an expression vector comprising a polynucleotide encoding a mutant eukaryotic initiation factor 5A1 (eIF-5A1) that contains a mutation at residue 50 of SEQ ID NO: 8; and
(b) an amount of small interfering RNA (siRNA), wherein the polynucleotide sequence of the siRNA will interfere the expression of endogenous eIF-5A but not the mutant eIF-5A1.

2. The method of claim 1 wherein the expression vector and siRNA are linked to a polyethylenimine (PEI) nanoparticle.

3. The method of claim 2 wherein the PEI nanoparticle is administered intravenously.

4. The method of claim 1 wherein one of the strands the siRNA comprises the polynucleotide sequence of 5′-GCUGGACUCCUCCUACACAdTdT-3′ and the opposite strand of the siRNA comprises the polynucleotide sequence of 3′-dTdTCGACCUGAGGAGGAUGUGU-5′.

5. The method of claim 1 wherein one of the strands the siRNA consists of the polynucleotide sequence of 5′-GCUGGACUCCUCCUACACAdTdT-3′ and the opposite strand of the siRNA consists of the polynucleotide sequence of 3′-dTdTCGACCUGAGGAGGAUGUGU-5′.

6. The method of claim 1 wherein the B cell lymphoma is selected from the group consisting of diffuse large B cell lymphoma (DLBCL), chronic lymphocytic leukemia (CLL), mantle cell lymphoma and follicular lymphoma.

7. The method of claim 1 wherein one of the strands the siRNA comprises the polynucleotide sequence of 5′-AAGCUGGACUCCUCCUACACAdTdT-3′ and the opposite strand of the siRNA comprises the polynucleotide sequence of 3′-dTdTUUCGACCUGAGGAGGAUGUGU-5′.

8. The method of claim 1 wherein one of the strands the siRNA consists of the polynucleotide sequence of 5′-AAGCUGGACUCCUCCUACACAdTdT-3′ and the opposite strand of the siRNA consists of the polynucleotide sequence of 3′-dTdTUUCGACCUGAGGAGGAUGUGU-5′.

9. The method of claim 1 wherein the substitution at K50 of SEQ ID NO: 8 is K50R.

10. The method of claim 1 wherein the polynucleotide encoding the mutant eIF-5A1 containing a substitution at residue 50 comprises the nucleotides 827-1287 of SEQ ID NO: 13.

11. The method of claim 1 wherein the polynucleotide encoding the mutant eIF-5A1 containing a substitution at residue 50 consists of the nucleotides 827-1287 of SEQ ID NO: 13.

12. The method of claim 1 wherein the expression vector further comprises a promoter that is active in B cells, wherein the promoter controls expression of the mutant eIF-5A1 containing a substitution residue 50 of SEQ ID NO: 8 in B cells.

13. The method of claim 1 further comprising administering bortezomib or lenalidomide to the human subject.

14. A method of treating a multiple myeloma in a human subject in need thereof comprising administering:
(a) an expression vector comprising a polynucleotide encoding a mutant eukaryotic initiation factor 5A1 (eIF-5A1) that contains a mutation at residue 50 of SEQ ID NO: 8;
(b) an amount of small interfering RNA (siRNA), wherein the polynucleotide sequence of the siRNA will interfere the expression of endogenous eIF-5A but not the mutant eIF-5A1; and
(c) an agent selected from bortezomib or lenalidomide.

15. The method of claim 14 wherein the expression vector and siRNA are linked to a PEI nanoparticle.

16. The method of claim 14 wherein the PEI nanoparticle is administered intravenously.

17. The method of claim 14 wherein one of the strands the siRNA comprises the polynucleotide sequence of 5′-GCUGGACUCCUCCUACACAdTdT-3′ and the opposite strand of the siRNA comprises the polynucleotide sequence of 3′-dTdTCGACCUGAGGAGGAUGUGU-5′.

18. The method of claim 14 wherein one of the strands the siRNA consists of the polynucleotide sequence of 5′-GCUGGACUCCUCCUACACAdTdT-3′ and the opposite strand of the siRNA consists of the polynucleotide sequence of 3′-dTdTCGACCUGAGGAGGAUGUGU-5′.

19. The method of claim 14 wherein one of the strands the siRNA comprises the polynucleotide sequence of 5′-AAGCUGGACUCCUCCUACACAdTdT-3′ and the opposite strand of the siRNA comprises the polynucleotide sequence of 3′-dTdTUUCGACCUGAGGAGGAUGUGU-5′.

20. The method of claim 14 wherein one of the strands the siRNA consists of the polynucleotide sequence of 5′-AAGCUGGACUCCUCCUACACAdTdT-3′ and the opposite strand of the siRNA consists of the polynucleotide sequence of 3′-dTdTUUCGACCUGAGGAGGAUGUGU-5′.

21. The method of claim 14 wherein the substitution at K50 of SEQ ID NO: 8 is K50R.

22. The method of claim 14 wherein the polynucleotide encoding the mutant eIF-5A1 contains a substitution at residue 50 comprises the nucleotides 827-1287 of SEQ ID NO: 13.

23. The method of claim 14 wherein the polynucleotide encoding the mutant eIF-5A1 contains a substitution at residue 50 consists of the nucleotides 827-1287 of SEQ ID NO: 13.

24. The method of claim 14 wherein the expression vector further comprises a promoter that is active in B cells, wherein the promoter controls expression the mutant eIF-5A1 containing a substitution residue 50 of SEQ ID NO: 8 in B cells.
